(19) United States
(12) Patent Application Publication
(10) Pub. No.: US 2024/0355467 A1
(43) Pub. Date: Oct. 24, 2024

(54) METHOD AND SYSTEM FOR MONITORING PRESCRIBED MOVEMENTS

(71) Applicants: HODA SALSABILI, West Lafayette, IN (US); MOHAMMAD HOSSEINI, West Lafayette, IN (US)

(72) Inventors: HODA SALSABILI, West Lafayette, IN (US); MOHAMMAD HOSSEINI, West Lafayette, IN (US)

(21) Appl. No.: 18/683,058

(22) PCT Filed: Aug. 11, 2022
(86) PCT No.: PCT/US22/40130

§ 371 Date: Feb. 12, 2024

Related U.S. Application Data
(60) Provisional application No. 63/232,357, filed on Aug. 12, 2021.

(51) Int. Cl. G16H 40/67; G16H 20/30

(52) U.S. Cl. CPC G16H 40/67; G16H 20/30

(57) ABSTRACT

This invention provides a method, apparatus, and system for real-time monitoring and tracking individualized physical therapy exercise plan and classes to remote users using artificial intelligence/machine learning. In one aspect, the present disclosure comprising providing information about prescribed individualized exercises and classes by a physical therapist that can be accessed via a digital communication network by a user at a remote location, sending digital video and audio content comprising prescribed individualized exercises and classes from a server to a personal digital device or a computer associated with the system, tracking and sending real-time feedback for the correct pattern of body movements via the system or a Tele-Physical therapist, providing a social platform for communication among users or users-physical therapist, and a summarized report of the biomechanical movements of the user.

### CROSS-REFERENCE TO RELATED APPLICATION

This patent application claims priority to U.S. Provisional Application 62/232,357 filed Aug. 12, 2021, the disclosure of which is considered part of the disclosure of this application and is hereby incorporated by reference in its entirety.

### FIELD OF THE INVENTION

This invention provides a systems, methods, and apparatus for monitoring and tracking individualized physical therapy exercise plan of remote users. In one aspect, the invention relates to a real-time monitoring tracking system and method utilizing artificial intelligence/machine learning for monitoring prescribed body movements to individuals and/or groups for real-time feedback and social communication among users.

### BACKGROUND

In the physical therapy industry, in home therapeutic exercises are commonly assigned to patients and individuals to help ensure and improve physical recovery when in-person therapy session is not available or used. Typically, adherence of patients to plan of care and exercises is very low, approximately 35%. Low adherence indicates the patients do not complete the recommended therapy sessions or do not perform exercises regularly at home or in the clinic. For physical therapist (“PT”), low adherence lowers profit for missing sessions and increases denied claims for lack of progress in the patients' recovery. For insurance companies, low adherence costs higher rate of rehospitalization and disability.

Some factors concluding to low adherence of patients are poor tracking of mental and physical pain and performance during and after exercises, lack of feedback or correct instruction, little motivation and social engagement, and lack of planned and various exercises for prescribed exercises. Often physical therapists or trainers print exercises on a paper, give verbal or video prescription of exercise or in the best scenario, they use some commercial cell-phone applications which has extensive limitations to monitor patient/trainee specifically out of clinic. These methods leave exercises for patients without any real-time biomechanical feedback analysis for correct performance, biofeedback for physiological progress or any objective report that summarize the progression of patient/trainee by exercises. Additionally, the limitation in the number and category of prescribed exercises does not allow the physical therapist to make a comprehensive exercise plan for the patient/trainee. An appropriate communication between physical therapist/trainer with the patient/trainee is also missing. For example, using tele-communication between patient and physical therapist and rating pain and performance in comparison with the group of people with similar complications for the prescribed exercises or attending an exercise class can essentially help tracking of pain and performance for patients. Moreover, the therapy environment is not often very interactive and social engagement with other patients is very limited that reduces the motivation of patients to adhere to their plan of care. An environment that a group of patients practice together with a social interaction capacity would solve the motivation needed for a better adherence.

A system monitoring the exercise at home or remote location that patients objectively see their progress, engage in social environment and become aware of their body performance during exercises can subsequently increase the adherence of patients to their plan of care and benefit them for a better recovery. It also let the physical therapist to follow the patient progress, increase the patients' recovery, and encourage the patients to attend all prescribed sessions in the plan of care effectively and objectively. Additionally, the system can help insurance companies to reduce the cost of non-adherence including higher rate of rehospitalization and disability in non-adhered patients to their plan of care and request for an objective progress note from the physical therapists.

There exists a need for a system and method that allows physical therapist/trainer to monitor and engage the patients/clients in a private and/or class and social environment to motivate the exercise performance at remote locations.

### BRIEF SUMMARY OF THE INVENTION

In one aspect, the present disclosure relates to a real-time movement tracking system for monitoring prescribed movements assigned to a user to provide real-time feedback during the performance of the prescribed movement in a remote location.

In another aspect, the present disclosure relates to a movement feedback system that can include a memory having one or more prescribed movement programs and/or classes. The movement programs can include a one or more movements and one or more movement parameters for each of the movements. The memory can also store a user profile for a user. In some exemplary embodiments, multiple users can have unique profiles and user identification assigned to their user profile. A processing means can execute instruction on a memory to perform various operations including but not limited to initiating a prescribed movement program containing at least one or more movements to be performed by a user, monitoring and recording the movements of the user utilizing a modeling algorithm with a camera, generating feedback based upon the movement of the user and analysis of the user movement, and communicating response feedback. The feedback can include any suitable feedback, including but not limited real-time feedback using artificial intelligence/machine learning from the system 3 comparing a measured movement parameter to a target movement parameter or real-time feedback provided by a tele-physical therapist, or generated real-time feedback using visual, auditory, haptic modalities. The modeling algorithm can utilize human pose estimation model for each movement that can be further enhanced using an artificial intelligence and/or machine learning engine.

In another aspect the present disclosure relates to a method for providing remote physical therapy session for a patient/user at their residence. Information/instruction can be communicated to a user device from the cloud and/or a server that contains exercises or movements that the individual was assigned to aid in a recovery plan. The system can monitor and gather information using artificial intelligence/machine learning pertaining to the performance of the user when conducting the exercises at home through visual data obtained by the systems camera and/or movement monitoring system. Information/data about prescribed individualized exercises and performance by the user of the exercises can be transmitted to an instructor and/or physical therapist that can be accessed via a digital communication network by a user at a remote location, sending digital video and audio content comprising prescribed individualized exercises. The system and method can additionally provide real-time feedback based upon the user's performance as the user is performing the prescribed movements. The system can generate a performance report and transmit the report to the user and/or the instructor upon completion of the prescribed exercise(s) based upon the performance of the user.

In another aspect the present disclosure relates to a method for providing group physical therapy classes from a server to one or more users through a personal digital device or to a computer associated with the system. The method and system can track and send real-time feedback for the correct pattern of body movements using artificial intelligence/machine learning via the system or a Tele-Physical therapist. The classes/instruction can similarly provide a social platform for exercising group of patients with similar complication and under the supervision of a Tele-PT.

In yet another aspect, the present disclosure is related to a method for providing a prescribed exercise routine live to a pre-determined user by providing information to a remote user about a pre-determined exercise routine established by a third-party that can be accessed via a digital communication network by the pre-determined user to which the pre-determined exercise routine corresponds. An interface can be provided that can include a graphical display and a camera, wherein the display can allow the pre-determined user to access and view the pre-determined exercise routine and the camera can be configured to record and/or monitor one or more movements of the pre-determined user while participating in the pre-determined exercise routine. Video, audio and/or audiovisual data can be obtained, transmitted, and/or recorded of the one or more movements of the pre-determined user. The obtained data of the one or more movements can then be analyzed with respect to a movement database and/or through artificial intelligence/machine learning methods for tracking patients performance and/or through one or more pre-determined movement parameters assigned to respective exercise for the pre-determined user. An assessment, score, and/or rating can be assigned to each of the recorded movements and based upon the assessment and/or analysis real-time feedback can be transmitted to the pre-determined user. Upon completion of the pre-determined exercise routine post-performance feedback can be provided to the pre-determined user. Additionally, the post-performance feedback can be transmitted and/or communicated to a third-party.

In yet another aspect, the present disclosure relates to a method for providing a live and/or archived exercise routine group courses to and a platform for social communication among remote users by first providing information to one or more remote users about one or more pre-determined exercise routines established by a third-party, and second, by providing social interaction among users that can be accessed via a digital communication network by one or more remote users. A user interface can be provided to one or more remote users that can include a display and a camera for each of the one or more remote users, wherein the display can allow remote users to access the pre-determined exercise routine and the social communication platform, and configured camera for recording and/or monitoring one or more movements of the remote users while participating in the selected exercise routine and courses. A user can select one or more pre-determined exercise courses for broadcasting on the display and initiate the exercise routine. The video of the one or more users performing the prescribed course can be received and analyzed by artificial intelligence/machine learning with respect to a movement database that can include goal parameters for the one or more prescribed movements individually specific to each the one or more users. Additionally, the video and/or audiovisual content obtained/recorded from the camera and/or device can be communicated to a third-party monitoring the one or more users performing the prescribed exercise routine. Real-time feedback can be provided to one or more of the users based upon the analysis of the system and/or by a third party instructor/therapist that obtains the recorded/live-stream video from the one or more users. Post routine/performance feedback can be provided and transmitted to one or more of the users that participated in the group session upon completing the exercise routine. The post performance feedback can additionally be communicated to a third-party. The interface can additionally allow social interaction among one or more users that can selectively follow or be followed by one or more of the other users, as well as communicate with one another.

In yet another aspect, the present disclosure relates to a method for providing an exercise routine and feedback to one or more users. A movement monitoring and feedback system can provide information to a remote user about one or more exercise routines that can be established by a third-party. The exercise routines can be accessed through a digital communication network by one or more pre-determined users. In some embodiments, the exercise routines can be assigned to a one or more users by the third-party. A user interface can be provided that can include a display and a video monitoring system, such as a camera. The display can allow a user to access the exercise routine and the camera can be configured to capture video data of the one or more movements of the user while participating in the exercise routine. The display can provide the user with a selected exercise routines for broadcasting on the display which can then be provided and communicated through the interface. Video data can be obtained of the one or more movements of the user through the video monitoring system as the user performs the movement. The video data can be analyzed by the system utilizing one or more databases including but not limited to a movement database and one or more pre-determined movement parameters assigned to the pre-determined user for the exercise. The system can determine and generate a rating of the performance of the recorded movements of the user with respect too the movement parameters and can further provide real-time feedback to the user based upon the performance of the movement. The real-time feedback can be provided by one or more of a remote telecommuting instructor through the system and an automated feedback response from the system. The system can provide a post-performance feedback report to the user and/or a third party.

### BRIEF DESCRIPTION OF THE DRAWINGS

FIG. 1A is a block diagram of an exemplary system of the present disclosure.

FIG. 1B is a block diagram of an exemplary system of the present disclosure.

FIG. 2A is a diagram of an exemplary embodiment of the method of the present disclosure.

FIG. 2B is a diagram of an exemplary embodiment of the method of the present disclosure.

FIG. 3 is an illustration of a user interface for a patient utilizing an exemplary embodiment of the present disclosure and social interaction with other users.

FIG. 4 is an illustration of a user interface for a patient utilizing an exemplary embodiment of the present disclosure for viewing schedule classes.

FIG. 5 is an illustration of a user interface for a patient utilizing an exemplary embodiment of the present disclosure showing a user's prescribed individualized classes and available group classes

FIG. 6 is an illustration of a user interface for a patient utilizing an exemplary embodiment of the present disclosure during an individualized prescribed class and performance of a prescribed movement that analyzed with artificial intelligence/machine learning.

FIG. 7 is an illustration of a user interface for a patient utilizing an exemplary embodiment of the present disclosure for the group class and social platform.

FIG. 8 is an illustration of a user interface for a patient utilizing an exemplary embodiment of the present disclosure showing progress illustrations for various prescribed exercises.

FIG. 9 is another illustration of a user interface for an instructor/physical therapist utilizing an exemplary embodiment of the present disclosure for prescribing exercises to individuals or patients.

### DETAILED DESCRIPTION OF THE INVENTION

The following detailed description includes references to the accompanying drawings, which forms a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present disclosure can provide one or more embodiments that may be, among other things a method, system, or computer program and can therefore take the form of a hardware embodiment, software embodiment, or an embodiment combining software and hardware. In one exemplary embodiment, the present invention can include a computer-program product that can include computer-useable instruction embodied on one or more computer readable media.

Computer-readable media include both volatile and nonvolatile media, removable and nonremovable media, and contemplates media readable by a database, a switch, and various other network devices. Network switches, routers, and related components are conventional in nature, as are means of communicating with the same. By way of example, and not limitation, computer-readable media comprise computer-storage media and communications media.

Computer-storage media, or machine-readable media, include media implemented in any method or technology for storing information. Examples of stored information include computer-useable instructions, data structures, program modules, and other data representations. Computer-storage media include, but are not limited to RAM, ROM, EEPROM, flash memory or other memory technology, CD-ROM, digital versatile discs (DVD), holographic mediator other optical disc storage, magnetic cassettes, magnetic tape, magnetic disk storage, and other magnetic storage devices. These memory components can store data momentarily, temporarily, or permanently.

The various components can include a communication interface. The communication interface may be an interface that can allow a component to be directly connected or communicatively connected to any other component or allows the component to be connected to another component over network. Network can include, for example, a local area network (LAN), a wide area network (WAN), cable system, telco system, or the Internet. In an embodiment, a component can be connected to another device via a wireless communication interface through the network, including but not limited to cellular mobile communication (such as 2G, 3G, 4G and 5G networks), Bluetooth Low Energy, WiFi, Near Field Communication (NFC), Ultra Wideband (UWB), or other radiofrequency means. Some exemplary embodiments of the assessments and tracking system of the present disclosure and method may be described in the general context of a computer-executable instruction, such as program modules, being executed by a computer. Generally, program modules may include routines, programs, objects, components, data structures, among other modules, that may perform particular tasks or implement particular abstract data types. The various tasks executed in the system may be practiced by distributed computing environments where tasks are performed by remote processing devices that are linked through communications network, which may include both local and remote computer storage media including memory storage devices and data.

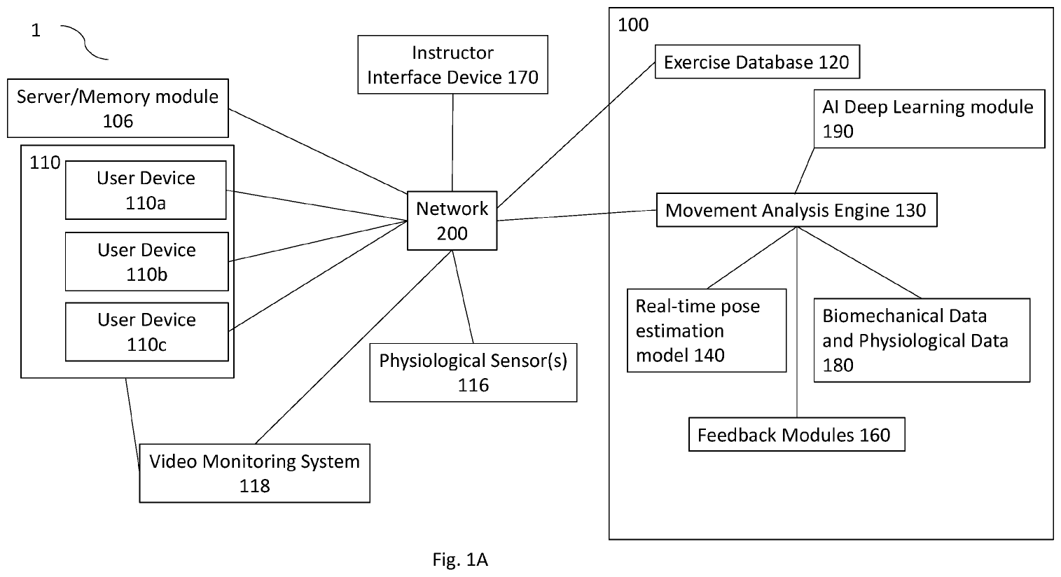

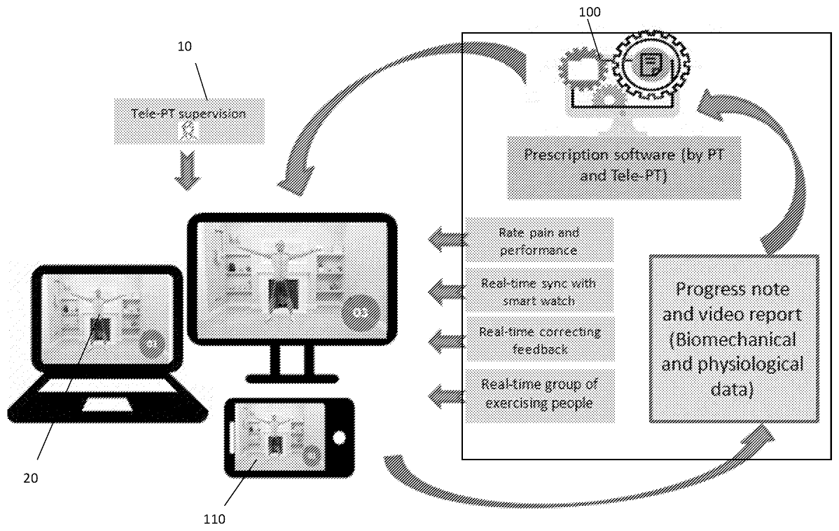

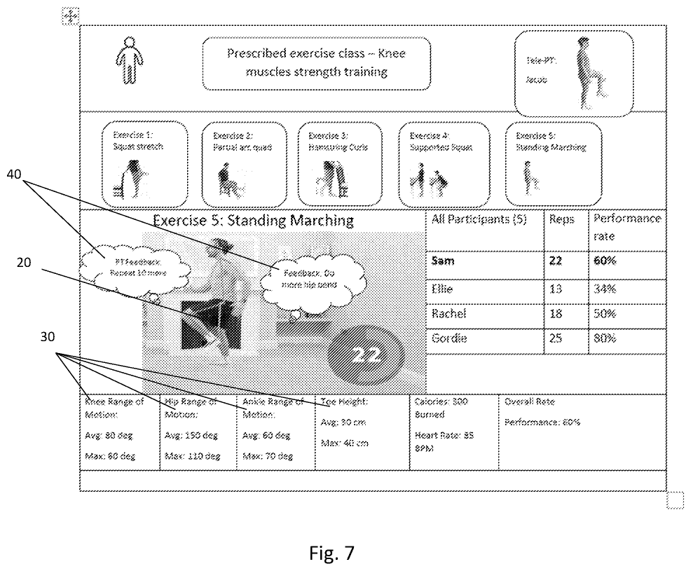

As shown in FIGS. 1A-B, in one exemplary embodiment of the present disclosure, a movement feedback system 1 of the present disclosure can include one or more software/application exercise database modules 120 for ordering prescribed exercises to one or more users. The exercise databases 120 can include a variety of categories of exercises for different groups of users and selects appropriate measures to report the progress in each exercise to the physical therapist/instructor. Each exercise can additionally include one or more movement parameters 30 which can be displayed to a user as shown in FIG. 7. A user specific goal measurement can be assigned to the one or more movement parameters for each of the related exercises assigned to the user. The various exercises, exercise programs, and corresponding movement parameters can be stored on a memory module which can include but is not limited to a physical memory or server, and customized into one or more pre-determined training modules/programs to be assigned by an instructor 10 for one or more specific users 20.

The system 1 can communicate with a manufactured hardware/or patient personal user device 110 such as cellphone, tablet or laptop. An integrated graphical display or separate graphical display can be communicatively coupled to the user device 110 can display the exercises and a video monitoring system 118 can collect video content including real-time biomechanical/physiological data as a user performs an exercise. The method and system of the present disclosure can provide real-time feedback 40 to a user and data through a movement analysis engine that can include a human pose estimation model 140 and image analysis by artificial intelligence/machine learning module 190. The system can then provide real-time auditory, visual, and vibrating tactile feedback during exercises to correct pattern of exercise performance.

The system can utilize any computing means such as cloud computing, server, 106, or computing through a user device 110. The computing means may include an address/data bus that is configured to communicate information. Additionally, one or more data processing units, such as a processor (or processors), are coupled with the address/data bus. The processor can be configured to process information and instructions. In one exemplary embodiment, the processor can be a microprocessor. Alternatively, the processor may be a different type of processor such as a parallel processor, application-specific integrated circuit (ASIC), programmable logic array (PLA), complex programmable logic device (CPLD), or a field programmable gate array (FPGA).

The memory module 106 can also include data storage units, such as volatile memory unit (e.g., random access memory (“RAM”), static RAM, dynamic RAM, etc.) coupled with the address/data bus, wherein a volatile memory unit is configured to store information and instructions for the processor. The computer system further may include a non-volatile memory unit (e.g., read-only memory (“ROM”), programmable ROM (“PROM”), erasable programmable ROM (“EPROM”), electrically erasable programmable ROM “EEPROM”), flash memory, etc.) coupled with the address/data bus, wherein the non-volatile memory can be configured to store static information and instructions for the processing means. In other exemplary embodiments, the computer system may execute instructions retrieved from an online data storage unit/memory such as in “Cloud” computing or virtual service. In some exemplary embodiments, the computer system can include one or more interfaces, such as an interface, coupled with the address/data bus. The one or more interfaces are configured to enable the computer system to interface with other electronic devices and computer systems. The communication interfaces implemented by the one or more interfaces may include wireline (e.g., serial cables, modems, network adaptors, etc.) and/or wireless (e.g., wireless modems, wireless network adaptors, etc.) communication technology.

The computing system can further be communicatively coupled to one or more other devices such as a tablet, smart phone, camera, personal computers, laptops, sensors, or wearable device, among others. In some exemplary embodiments, the system can provide information to a user or instructor via a graphical display that may be incorporated into a user device 110 or communicatively coupled to the device 110. The graphical display can be any suitable means including but not limited to a mobile phone, tablet, glasses, smart watch, projector, television, video monitor or any other device capable of displaying video content. In some exemplary embodiments, the user device can include a processing means, a transceiver for communications to a network 200, and a graphical display for the user. In other exemplary embodiments, a server can function as a processing means for analysis of any audiovisual data captured during the performance of a movement by a user.

The processing means and computer of the present disclosure is one exemplary embodiment in accordance with the system of the present disclosure and is not meant to be a limiting example a computer system. The present disclosure provides a computer/processing system represents a type of data processing analysis that may be used in accordance with various aspects described herein. The processing means and/or computers system can be used to implement computer-executable instructions, such as program modules, being executed by a computer by one or more processors. In some exemplary embodiments program modules include routines, programs, objects, components and/or data structures that are configured to perform particular tasks or implement particular abstract data types.

The system of the present disclosure can be utilized to distribute through one or more computing environments, where tasks can be performed by remote processing devices that are linked through a communications network, or such as where various program modules are located in both local and remote computer-storage media including memory-storage devices, virtual databases, or memory modules/servers. Similarly, the system can record and process tasks being executed via one or more devices having a camera to collect audio and/or video data. The databases can include a library of exercises and/or movements that can be used to support a variety of categories of training needed for different groups of patients/trainees that any intended exercises by the physical therapist/trainer can be prescribed.

As shown in FIGS. 1A-B, the system can include one or more computing devices, such as a server 106, user device 110, or instructor interface device 170. The computing devices can include a processor means and/or a memory. In some exemplary embodiments, the computing device can be communicatively coupled to one or more networks 200. The device can additional be connected to one or more servers/cloud storage databases 106 and application databases 100 via the network 200. Additionally, the system can designate, assign, and or store one or more users which can include patients, evaluators, and/or instructors. In some exemplary embodiments, the system 1 can have one or more physical therapist instructors 10 assigned to one or more users/patients 20. Each of the users 20 can have a pre-determined device code and account number for communicating with the system 1. The system can further include a video monitoring system 118 such as a camera, video camera, smart phone, or other device that can capture images and/or videos of one or more users performing various exercises or prescribed movements from the instructor/physical therapist. In some exemplary embodiments, the video monitoring system 118 can be part of the user device 110.

As shown in FIGS. 1A-B, the movement feedback system can be communicatively coupled to various application databases 100, including but not limited to a movement analysis engine 130 that can include one or more feedback modules 160, artificial intelligence/Machine learning modules 190, or other programs, models, or algorithms for assessing and tracking movement of a user 20 to make an assessment of their performance of the prescribed rehabilitation program or prescribed exercises. The movement feedback system can provide real-time feedback to the remote user as the user is performing the prescribed or selected exercises. The feedback modules 160 and/or programs can include a rate pain and performance module, a real-time sync to timer program, a real-time correcting feedback module to provide to one or more users a real-time analysis of individual exercise. In some exemplary embodiments, the movement feedback system 1 can obtain and utilize biomechanical data and/or physiological data 180 and process the data by utilizing a human pose estimation model 140 of the user movement video content transmitted from the video monitoring system 118. Additionally, one or more analysis programs included but not limited to deep learning and artificial intelligence methods 190 can further generate feedback to be transmitted to the user in real-time. The system and method can be improved and automated through various techniques including computer vision, artificial intelligence, deep learning, and/or sensor (IMU) data in addition to text data: questions asked by a human agent, or an artificial intelligence bot-based on sent images, videos and previous answers as well as answers by the consumer from a smartphone and/or other device.

The movement feedback system of the present disclosure can utilize data obtained to assess and analyze the performance of the remote users without an evaluator physically present. Similarly, the movement feedback system 1 of the present disclosure can provide biomechanical and physiological data back to an instructor/therapy professional and/or the user for further analysis and for real-time feedback. The system can additionally provide a score/rating to the evaluator and/or the user. The rating can be provided on an exercise by exercise, repetition by repetition, and or routine by routine basis to provide detailed information to the evaluator as well as the user. Based upon the ratings, the system can generate new routines or exercises for the user. Similarly, an evaluator can determine to revise or generate a new routine for a user based upon the report. The rating can be stored on a memory module 106 for further comparison in subsequent sessions/programs performed by the user. The score or rating can include any suitable rating system (i.e., scale of 1-10, 1-100, 5 star rating, etc.). In some exemplary embodiments, a rating can be generated for each of the prescribed movements individually and/or for the entire routine/session of movements. The rating and/or analysis generated by the system can be utilized to determine whether or not and/or what type of real-time feedback to provide to a user. The rating can similarly be utilized to compare an individual user with other users having similar injuries or that have participated in similar movements/exercises.

The rating can be based on one or more metrics including number of repetitions completed, number of repetitions completely correctly, assessment of measured movement parameter versus the goal movement parameter, etc. Real-time feedback can be generated and provided to a user and/or group of users in the class using artificial intelligence and/or machine learning during the performance of the one or more prescribed movements. This feedback can help correct and/or ensure that the user is properly performing the prescribed exercise during the course of performance in real-time. In some embodiments, the real-time feedback can be generated and determined based upon measured movements of a user as they are performing prescribed movements against a predetermined goal movement parameter for the prescribed user. At the end of each specific exercise and/or at the end of the entire routine prescribed, a user can additionally provide a pain rating that can be communicated and/or stored for review by a third party. Similarly, the pain rating can also be included in the final post-performance feedback report generated by the system 1.

An instructor 10 can create an exercise or rehabilitation program/module in the exercise database 120 that can then be assigned or prescribed to a user 20. The user 20 can access the program through a device 110 communicatively coupled to a network 200 by utilizing the pre-determined login information assigned to the individual user 20. In some exemplary embodiments, the device 110 can be a personal computer such as a tablet, laptop, phone, or other device, or can similarly be a specialized device provided by the instructor to the user. The system can then initiate the rehabilitation program corresponding to the applicable user. In some exemplary embodiments, the system can further include one or more to display instructions to the user and or obtain additional data, such as physiological data. In some exemplary embodiments, the secondary device can be a smart watch or additional sensor devices or monitoring straps. The physiological data can include but not limited to heart rate, blood oxygen level, step count, among others. The video monitoring system 118 can then monitor and track the movement of the user as the user performs the one or more prescribed movements. The system can utilize the video content/data obtained from the video monitoring system to generated real-time feedback to the user as they are performing the movements. In some exemplary embodiments, the system can utilize a pose estimation module to compare the obtained data to the pose model and can additionally be compared with the prescribed parameters set forth for the particular exercise for the specific user. The artificial intelligence and human pose estimation module can utilize any suitable methods including human pose estimation, API processing, and blaze pose modeling among others. The video monitoring system 118 can obtain real-time biomechanical data during the exercises and can synchronize with a physiological device/sensor 116 to collect the real-time physiological data in tandem with the video and/or biomechanical data. The various data and analysis of the movements by the system can be utilized to provide the real-time feedback to a user or a plurality of users simultaneously. Additionally, in some exemplary embodiments, the data and movement analysis can be utilized to provide a rating to the user for the prescribed movement and/or for the performance of the entire routine.

In one exemplary embodiment, a user/patient can first meet with an instructor/therapist for an initial assessment to establish a movement routine that can include one or more prescribed movements. Each of the one or more movements can have an initial measured parameter associated with the movement, which can be used as an initial movement parameter(s) for the prescribed movement. Additionally, a goal movement parameter can be assigned to the movement for the prescribed user 20. In some embodiments, as a user progresses, the instructor and/or system can revise or reassess the user's goal movement parameter. As the user performs the movements remotely, a video monitoring device 118 that is communicatively coupled to a user device 110 can obtain and analyze the user performing the prescribed movements.

While a user performs the prescribed exercises initiated by the exercise/rehabilitation program, the system can provide real-time feedback to the user through the device during and/or after each repetition of the prescribed exercise or movement required. In some exemplary embodiments, the user feedback can be any suitable feedback including but not limited to auditory, visual, and/or vibrating/tactile feedback during the performance of the movements and based upon the biomechanical data and analysis obtained by the system. Additionally, an evaluator can provide real-time feedback as well. Real-time biomechanical data can include both biomechanical movement data and physiological data related to each exercise performed by a user. The feedback can be transmitted back to the user through a display device, secondary wearable device, or any other suitable device. Similarly, in some instances the device can be communicatively coupled to a separate display such as a monitor or television, which can provide the auditory and/or visual feedback. Additionally, the device can be communicatively coupled to a smart speaker or other device to provide auditory feedback as the user performs the prescribed exercise. The system can further provide the physiological data in real time to the user, including but not limited to energy consumption, health condition, and other physiological data.

In some exemplary embodiments, the system can provide real-time tele-communication with a virtually located instructor 10, which can include but is not limited to a movement evaluator, trainer, physical therapist, occupational therapist, or any other third party for supervision and feedback as well based upon the movement execution by the patient/user 20. An instructor 10 can be assigned to or select a group class. Alternatively, the instructor 10 can select to be a participant/instructor in one or more prescribed individualized classes of a user 20. In some exemplary embodiments, the tele-instructor 10 can be required for group instruction classes but may not be required for individualized remote sessions. Additionally, in some exemplary embodiments, a user 20 can provide a rating of the instructor 10 that was present during the prescribed group class. The instructor 10 can communicate to the user through a device 170 and/or a separate interface such as a phone, tablet, or monitor.

In various exemplary embodiments, the movement feedback system can also include group classes in the exercise database 120 and further operate as a social platform that may be communicatively coupled with a plurality user devices 110. The user devices 110 can be in communication with a video chat platform and/or a video content distribution network that receives audio and/or video content from one or more of the user devices 110 to allow for communication between the various users in the group class or about the exercises as well as instructor 10. The networked social platform and methods may include multi-directional communication and data transfer capabilities that allow video, audio, voice, and data sharing among all users 20 through there user devices 110 and/or to instructors 10 and their interface 170. The system can further initiate and utilize virtual rooms for group exercising/therapy of patients/users 20 that may require the same or similar treatments and/or movements. Additionally, the system can provide a rating and/or score based upon each user's 20 performance. The rating can be compared to a stored database of ratings of other patients/users that have carried out similar movements and/or programs. The system can provide user feedback based upon these comparisons. In some exemplary embodiments the system can prescribe a recommended rehabilitation or movement program based upon the users need or injury diagnosis.

The movement feedback system of the present disclosure can then identify and select one or more specific exercises or prescribed movements to have a summarized report containing the assessment of the performance of the prescribed movement based upon the biomechanical and physiological data obtained during the performance by the user 20. The report can be used by the system to provide performance feedback to the user 20 and/or instructor(s) 10. The feedback report can then be utilized as a metric for future performance evaluation by the instructor 10, system 1, and/or user. In some exemplary embodiments, the report can include biomechanical analysis of exercise movement parameters, including but not limited to joint angles, displacement of body joints, the speed of joints' movement for each exercise among other biomechanical data metrics to which the system can provide a rating. In some exemplary embodiments, the report can include physiological analysis of each exercise performance includes but not limited to heart rate, number of steps, blood pressure, blood oxygen level, energy exertion, energy consumption among other physiological data metrics. The exercises and or movements can include any suitable movements/exercises required by an instructor including, but not limited to gait training exercises, strength training exercises, stretches, plyometrics, active assisted, balance training, mobilization, isometric exercises, among others.

The system of the present disclosure can archive and/or transmit or provide a summary feedback report to an instructor 10 to access prior to the next session to aid in ensuring that the user 20 makes particular changes to their movements or to ensure that the user is properly performing the movements/programs when at their house or remote outside of a rehabilitation or training facility. The instructor 10 may then use the feedback reports to assign or change the prescribed program in the exercise database 120 for the user or determine what movements may need to be directed and/or monitored during an in-person therapy/instruction session. Similarly, the reports and/or video data can be utilized to determine if movement goal parameters need to be changed, reassessed, or provide data for the instructor to focus on various aspects of the prescribed routine as it is being performed by the user 20. In some exemplary embodiments, the movement feedback system can provide in real-time and/or at the completion of a exercise program, a biomechanical analysis and/or movement parameters of each prescribed exercises/movements. The system can then generate recommended movements or generate programs in the exercise database 120 based upon a user's feedback reports and data from previous exercise sessions. Alternatively, an instructor can manually review the results and adjust the program accordingly.

The system 1 can additionally measure and compare the movement measurements in real-time of the user to the prescribed goal movement parameters for the prescribed movement. This comparison can be utilized by the system to generate the real-time feedback to the user 20 and similarly can be used to rate the performance of the movement by the user 20. Additionally, the system can track and compare the progress of a user's improvements in a prescribed movement based upon the one or more movement parameters monitored.

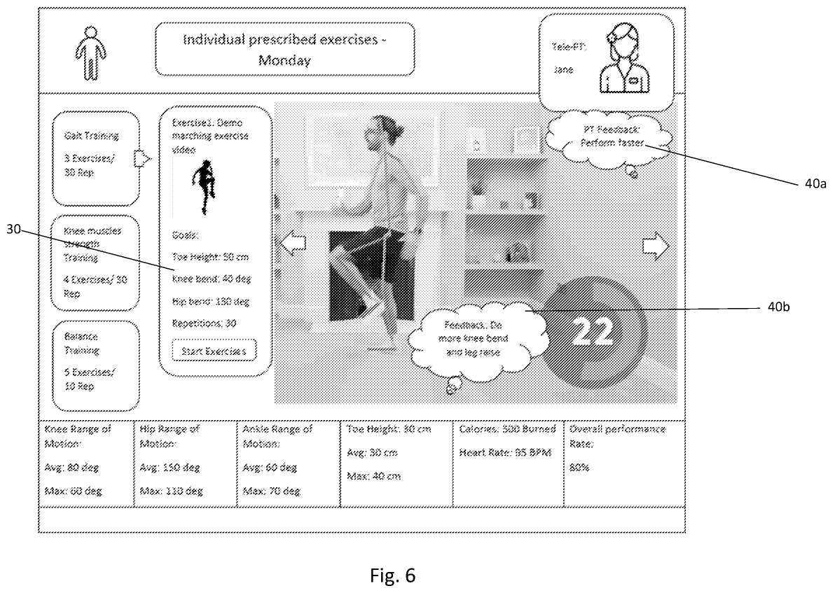

In some exemplary embodiments, they system can be communicatively coupled to secondary physiological sensors 116, such as a patient's smart watch or health monitor to monitor and give a report for physiological analysis of each exercise performance, which can include but is not limited to the heart rate, number of steps and energy consumption. The sensors 116 can additionally be utilized to monitor biomechanical movements as well. The system can utilize the audio/visual data obtained during the monitoring of the user performing the prescribed program separately or in conjunction with any obtained physiological data obtained. Additionally, the system 1 can be utilized for in person physical therapy sessions, which can be recorded and utilized at home by the user 20. The system can provide side by side display of the pre-recorded in person training and guidance corresponding to the movement initiated by the system for the at home therapy session as shown in FIG. 6. A user device 110 or a separate graphical interface can display one or more physiological readings from the secondary device configured to obtain the physiological data. The movement feedbacks system 1 of the present disclosure can further communicate during and after exercises with a virtual instructor 10, such as a Tele-PT, for a higher quality of supervision and feedback if requested or required.

Additionally, the system 1 can provide post-performance feedback and/or a summary report based upon the performance of each individual prescribed movement and the entire routine as a whole. A Tele-PT/instructors can also modify and prescribe exercises for the future or current session of exercising. Additionally, the data obtained of the remote users prescribed routine can be transmitted to a third party, such as a trainer or physical therapist for future review, further analysis, and or archived. The system can record and store the video and/or communicate the video in real-time to a third party. In some exemplary embodiments, one or more virtual rooms for group exercising of patients/users with implementation of a tele-PT/trainer. The synchrony between multiple patients and a therapist can be addressed in a virtual class exercising. The virtual group classes can allow for a social dynamic and ability of each of the remote users to communicate with the other remote users within the group class during the class. Additionally, the remote users can follow or be followed through a social network to communicate or track each other when not within the group class.

In some exemplary embodiments, the system of the present disclosure can also initiate a question or poll to a user after performing each individual movement and/or the entire therapy program. The poll can include pain rating questions as well as other feedback questions. The system can store and utilize the subjective rating of pain after each exercise and biomechanical and physiological data from patients performed each exercise to generate one or more feedback reports/summaries for review. As previously recited, the feedback report can then be transmitted to the instructor and/or the user. In accordance with the prescribed programs and/or program goals set by physical therapist/instructor and the population of patients performed in each exercise, provides rating of patients' performance and pain in comparison to patients with similar complications.

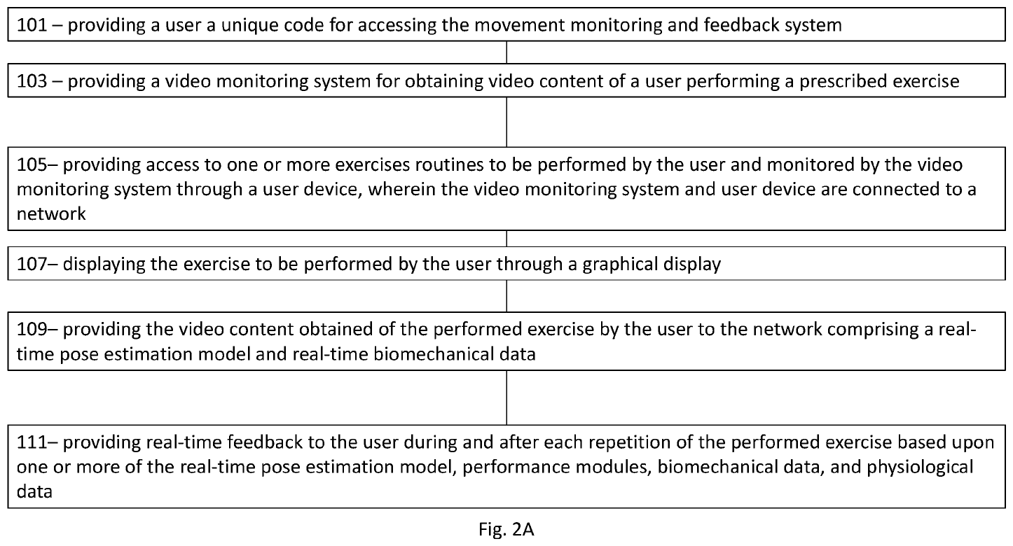

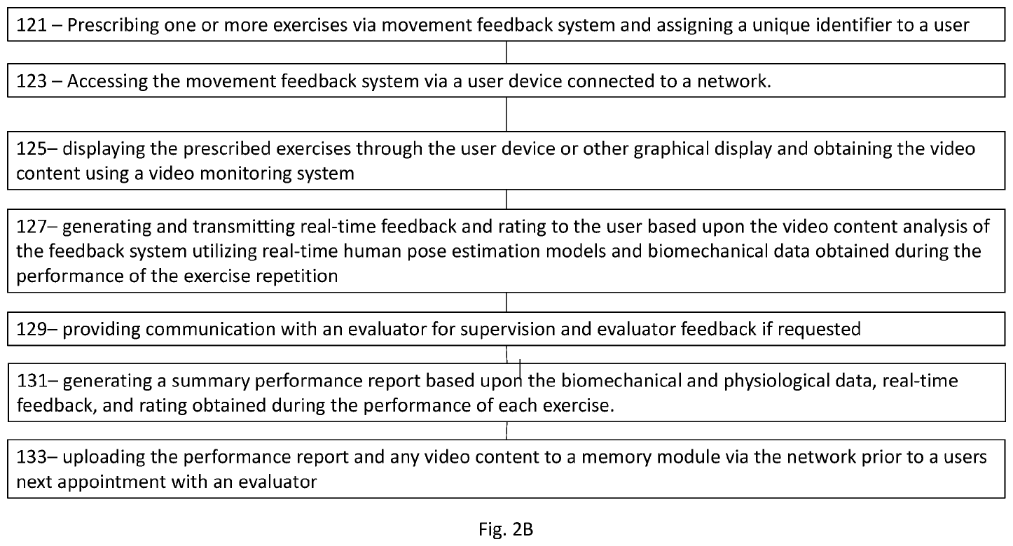

As shown in FIG. 2A, the present disclosure further provides a method for utilizing a movement feedback system of the present disclosure. The method can include first providing a user a unique code for accessing a movement feedback system previously described in the present disclosure (step 101). In some exemplary embodiments, the movement feedback system can be accessed or utilized via network 200 or through a downloadable app on a user device 110. A video monitoring system 118 can be provided for obtaining video content of the performance of the prescribed movements by one or more users (step 103). In some embodiments, a user device 110 can function as the video monitoring system and a graphical display. In other embodiments, the user device, video monitoring system, and graphical display can be separate apparatuses or a combination of one or more of the various apparatuses each of which can be communicatively coupled to the other and/or to the network.

The user can then be provided access to one or more exercises routines to be performed and monitored by the video monitoring system (step 105). In some exemplary embodiments, multiple users can be provided access to exercise routines of an exercise database. Additionally, the exercise routines can have an established time prescribed for their performance and multiple users can perform the exercises simultaneously in a virtual group class. Alternatively, exercise routines can be selected at any time to be performed by a user. In some exemplary embodiments, users can communicate with each other during the performance of exercise during the group class setting.

The movement feedback system can display the exercise to be performed by the user through a graphical display (step 107). This can provide a user with a real-time guide of how to perform each exercise during the execution of the exercise movement. The video content obtained by a video monitoring system during the performance of each repetition of the exercise can then be obtained and analyzed using one or more application databases 100 including but not limited to a movement analysis engine/algorithm that can include a real-time pose estimation model, real-time biomechanical data, and physiological data (step 109). The data can be transmitted over the network to a server or other device for processing. Real-time feedback can then be provided to a user during and after each repetition of the performed exercise based upon the analysis of the video content by the movement feedback system 1. The feedback can include any suitable information, including but not limited to performance rating, audiovisual correction feedback, number of repetitions, performance rating, heart rate, calories burned, range of motion rating, lift height, depth, speed of movement, and joint displacement among others. Additionally, the system can provide a performance data of each of the users in the group class during the class.

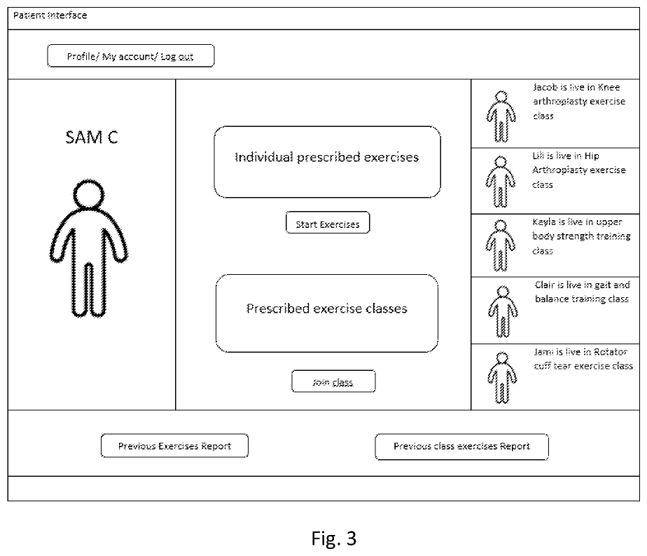

FIGS. 3-9 illustrate various graphical user interfaces that can be used by a user and/or an instructor when utilizing the system and providing feedback to the user when utilizing the system and method of the present disclosure. The user interface may present a variety of screens to the user 20, which the user 20 can move among to manage their experience, including selecting prescribed exercise routines, group classes or sessions, historical performance data and report, social communication and comments about an exercise or class, and arranging how such information is displayed on their system. Additionally, the interface can overlay information including performance information of a user or group of users communicated during the exercising, prescribed exercise movement and target information, real-time feedback, as well as the live video of the user. The user interface can allow a user to quickly access and move through various screens to access the users desired information or program including through touchscreen access.

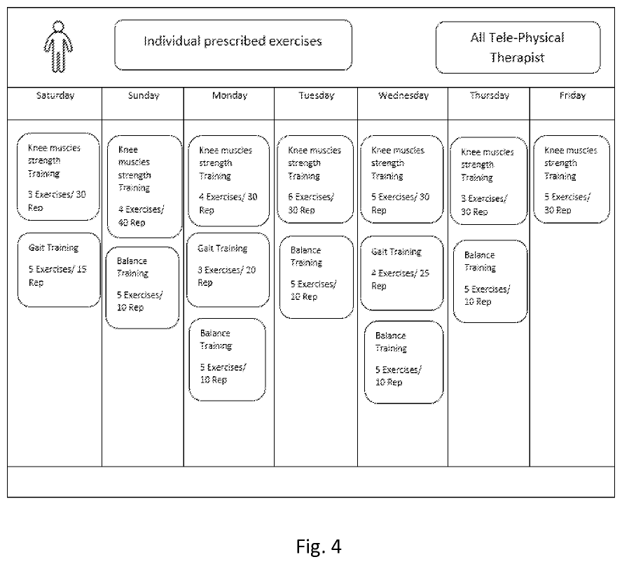

The user interface can provide a home screen that can be similar to that shown in FIG. 3 which can provide direct access and or links to the users account information and specifically prescribed individual courses and/or group courses that may be prescribed or that are available to the remote user. The home screen can also provide links to previous course performance information and/or reports and the commented feedback of our users left as a social interaction. As recited, the system can implement both individualized prescribed exercise programs/routines assigned to the user as well as live group courses that may be available, recommended, and/or prescribed to the user. The group courses can additionally include social networking information and communications between various users accessing the same group class or shared exercises. When a user selects the various class options, the user interface can be displayed in various formats including a list, calendar type (FIG. 4), or any other suitable format that provides the prescribed individual classes and/or the various available group classes and what dates those classes are either prescribed or available. The user interface can similarly allow a user to sort the programs based on various parameters including but not limited to type of training, group lessons, tele-physical therapist attended, date, time, etc.

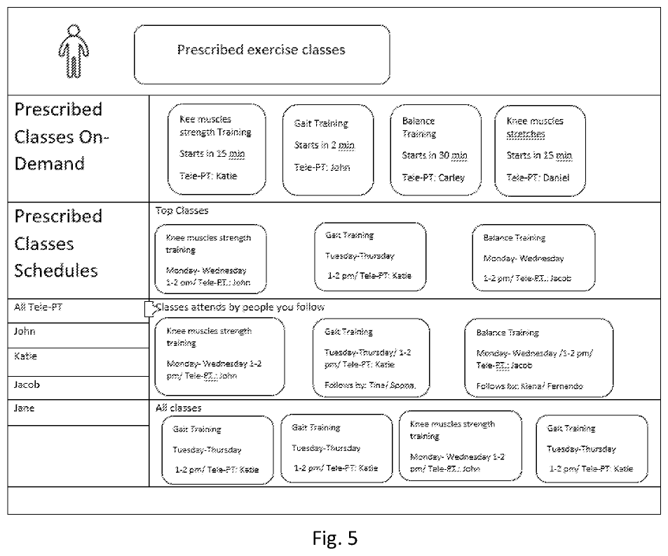

FIG. 5 illustrates a display provided by the interface that can provide various prescribed routines/classes to the user based upon their prescribed movements and routines as well as in relation to the social network and the classes that other similar users that they follow may be either signed up to attend or may have historically attended. These can include the assigned individual prescribed classes, assigned, prescribed, or related group classes, classes/routines that are attended by other users followed or networked by the user, and all classes available. Similarly, the interface can provide a list of classes that are available wherein a Tele-physical therapist/third-party instructor will be available and attending the class/session. User lists and information may be accessed, sorted, filtered, and used in a wide range of different ways. For example, other users can be sorted, grouped and/or classified based on any characteristic including personal information such as age, gender, weight, or injury type, recover program classification, similar classes assigned/prescribed. Additionally, in some exemplary embodiments, a user can rate and/or follow the tele instructor that conducts the prescribed group courses. Similarly, the system can provide notifications to a user when a user or instructor that is being followed has signed up for a group course or signed up to be the instructor for a particular course.

As shown in FIG. 6, the system can monitor the user's movements during the performance of a prescribed movement and/or exercise. The system can provide real-time feedback 40 based upon the video data obtained based upon the movement analysis algorithm to determine if the user is carrying out the prescribed movement within a prescribed threshold for the specific user. The feedback can be auditory, visual, and/or tactile feedback to inform the user of the prescribed feedback. Additionally, and/or alternatively, a Tele-physical therapist can provide real-time feedback 40a to the user based upon the system scoring and or visual assessment by the instructor. In some exemplary embodiments, each one or more prescribed movements can have goal parameters 30 assigned to the movement to ensure progress and or successful completion of the movement by the user. The system can display real-time assessments/feedback which can include feedback, ratings, and related measurements for the various prescribed movements. Additionally, during the performance of the prescribed class and related assigned movements, the interface can display information regarding the prescribed movement, including but not limited to reps required, type of movement, number of exercises, target/goal movement parameters, automated generated feedback by the system, feedback provided by a tele-physical therapist/instructor, demo videos, audio, and or images illustrating proper form of the exercise and real-time completion of the prescribed program.

FIG. 7 illustrates an exemplary embodiment of a user interface for a group class that can include a list of the various exercises/movements included in the group class, participants of the class, reps completed by each participant, performance rating related to each participant, and the target and average movement parameters of the individual user participating in the group class and their individualized goals/target measurements as it relates to their prescribed movements. Similarly, if an exercise is not applicable or may not be part of the individual's prescribed movement or does not apply to the prescribed routine related to the individual users program assigned by a third party trainer/physical therapist the user can be instructed or it may be displayed to skip or not-participate in a particular exercise/movement.

As previously recited, the system can generate a post-performance feedback report. The feedback report can provide illustrative and/or free-word assessment based upon the user's performance of each of the one or more movements prescribed to perform in one session, multiple sessions, and for selection of prescribed exercises. For instance, the feedback report can state that user only fully completed “x” number of repetitions of exercise 1 and out of the completed repetitions only “y” were correctly performed. Additionally, in some exemplary embodiments, a performance rating can be assigned to the user based upon the user's execution of the one or more movements comparing to other users with similar condition. The user may also provide additional feedback upon completion of the one or more exercises or at the end of the routine, such as pain associated with the one or more prescribed movements.

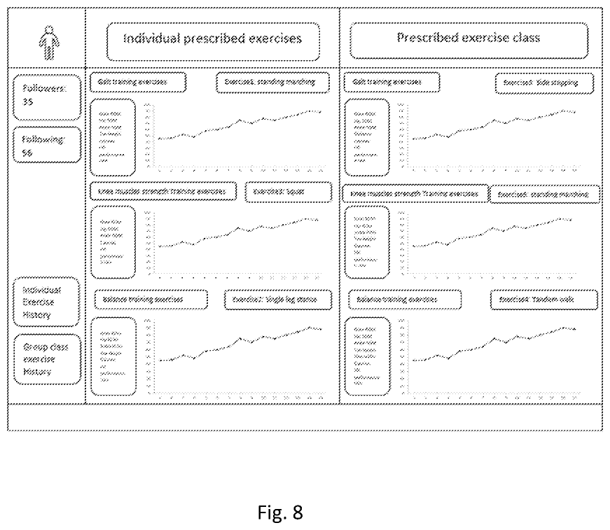

The user interface can display progress report(s) and historical performance data to a user via a display device. FIG. 8 is an exemplary embodiment of a display illustrating progress by a user over time based upon each of the prescribed exercises and the baseline or initial movement measurement and the current movement parameter or measurement related to the prescribed movement/routine. The baseline measurements can be stored utilizing the memory module and accessed through the network. The baseline measurements can be established on a per user per exercise basis. In some exemplary embodiments, the baseline measurements can be established using a contour base calibration based upon the individual user's personal information including but not limited to height and other body measurements. The calibration/baseline can be set for each of the prescribed exercises/movements to be performed by the user. The progress data can be displayed in any suitable manner including graphical illustrations of the improvements in movement over a period of time.

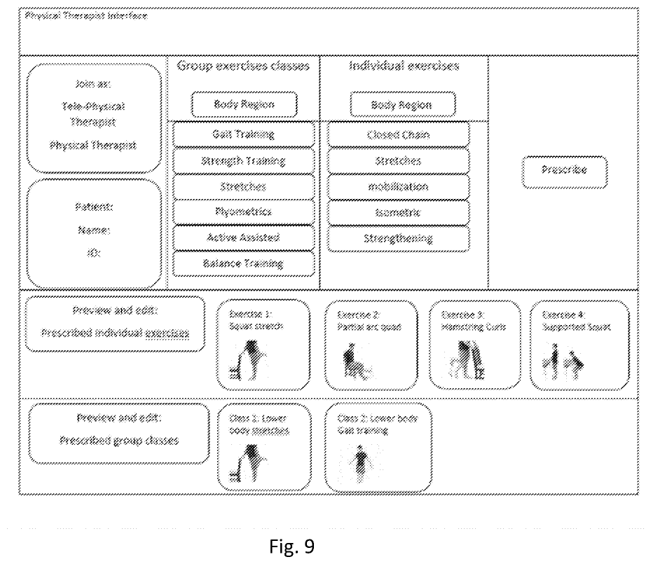

Additionally, the user interface of the system can be configured for a physical therapist, instructor, trainer, or other third party to design, assign, and implement prescribed activities to various users of the system. As shown in FIG. 9, the instructor interface can provide a display for the instructor to select a class or group class to join to provide real-time instruction to one or more users and communicate in a social platform with the users about the class or exercises or the related experiences and comments. The interface can display to the instructor the audio and/or visual data recorded/captured by the camera of the system obtaining the audiovisual and/or video data from the one or more users.

Additionally, the system can provide the same or different feedback to the instructor that it provides to the one or more users. In some embodiments, the system can provide different real-time feedback to the instructor to inform them of the one or more user's performance as the users are performing the various movements and/or exercises prescribed during the session. The interface can additionally provide user information including patient ID, name, injury type, surgery type, recovery class, age, gender, and/or other statistics. Similarly, the interface can allow the user to select and assign various exercises/movements to be prescribed as well as corresponding movement parameters/thresholds/goals for the prescribed user for the prescribe movement. The instructor can preview and edit developed exercise routines/programs prior to submitting them to a prescribed user. If a user selects a group course, the system can access the related movement parameters for specific user's goal parameters. If a user does not a prescribed goal parameter for a movement or is not assigned a movement included in a group course, the system can provide a notice to the user not to perform the exercise of that group session.

In some exemplary embodiments, the system of the present disclosure can provide multimedia inputs and outputs for social interaction among users, or with physical therapist about streaming or archived prescribed programs content that can include but not limited to rehabilitation workout routines or exercise movements, socially networked audio and video chat, networked performance metrics and competition capabilities, along with a range of rating features. The system can utilize a user interface that can be communicated to a user via a display or device, including but not limited to a computer, tablet, phone, or other device. The user interface can be used to access user information, login, logout, live content for group lessons, and or individualized prescribed routines or classes assigned to the specific user. User information may be displayed in variety of formats and can include the historical and current performance information and user account information, social networking links, and ratings, etc. The user interface can also be used to access the system to update profile information and manage account settings which can include information setting standards, social network information, control settings, among other items. In the social platform, the users are able to see the following friends, followed friends, chat box, commenting and sharing video or pictures, interact with instructors, observe performance and ratings of others, as well as other social features. Additionally, the user interface may display a progress percentage based upon the number of courses completed by the user in relation to the amount of courses prescribed to the user and the performance rate or relative rating to the other users with similar condition. Similarly, the progress rating may be based upon the user's mobility and/or performance of movements in relation to the target movement parameter.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. A movement real-time feedback system, comprising:
a memory having one or more prescribed movement programs and/or classes, wherein the movement programs can include a user profile for a user, one or more movements and one or more movement parameters wherein the memory is communicatively coupled to a network;
an interface that comprising a display and a camera, wherein the display allows user to access the movement programs and the camera is configured to capture assessment movement video data of the one or more prescribed movements of the user while participating in the prescribed movement programs, wherein the interface is communicatively coupled to the network;
one or more physiological sensors communicatively coupled to the network configured to obtain physiological data from a user;
one or more processing means to execute instruction on a memory to perform the following operations:
initiating a prescribed movement program containing at least one or more movements to be performed by a user;
providing a user a unique code for accessing the apparatus;
providing information to a remote user about one or more exercise routine established by a third-party that can be accessed via a digital communication network by the pre-determined user to which the pre-determined exercise routine corresponds;
providing an interface including a display and a camera, wherein the display allows user to access the exercise routine and the camera is configured to capture video data of the one or more movements of the user while participating in the exercise routine;
receiving from the user a selected exercise routines for broadcasting on the display;
providing the selected exercise routine and communication through the interface;
receiving video data of the one or more movements of the pre-determined user;
analyzing the one or more movements with respect to a movement database and one or more pre-determined movement parameters assigned to the pre-determined user with a modeling artificial intelligence guided algorithm;
monitoring and recording the movements of the user utilizing a modeling algorithm with a camera;
generating feedback based upon the movement of the user and analysis of the user movement;
communicating response feedback to the user wherein the feedback includes at least one of the following:
generated real-time feedback using artificial intelligence from the system comparing a measured movement parameter to a target movement parameter, or
generated real-time feedback using visual, auditory, haptic modalities;
communicating the post-performance feedback to the third-party.

2. The system of claim 1, a secondary device for monitoring physiological metrics of the user.

3. The system of claim 2, wherein the modeling algorithm comprises a human pose estimation model for each movement using artificial intelligence-guided modelling algorithm.

4. The system of claim 2, wherein the summary of the performance transmits to a third-party via a network.

5. The system of claim 2, wherein the memory can store one or more baseline measurements for each movement for the user and one or more prescribed measurements parameters for each movement.

6. The system of claim 5, wherein the feedback of an exercise performance includes at least one of the following:
range of motion, lift height, depth, speed of movement, or joint displacement.

7. The system of claim 5, further comprising one or more physiological sensors, wherein the physiological sensors monitors at least one of the following:
heart rate, energy exertion, number of steps, blood pressure, and blood oxygen.

8. The system of claim 5, further comprising a social network communicatively coupled to the network configure to allow users at least the following:
communication between users, exchanging comment, sharing video and picture, interacting with the instructor, and observing the performance and ratings of other users.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. The system of claim 1, wherein the exercise routine is prescribed to a user by the third-party

22. The system of claim 3, wherein the video data provides real-time biomechanical data.

23. The system of claim 22, wherein the human pose estimation model, real-time biomechanical data, and physiological data are used to generate the real-time feedback and artificial intelligence-guided written feedback to the user.

24. A movement real-time feedback system, comprising:
a memory having one or more prescribed movement programs and/or classes, wherein the movement programs can include a user profile for a user, one or more movements and one or more movement parameters, wherein the memory is communicatively coupled to a network;
an interface that comprising a display and a camera, wherein the display allows user to access the movement programs and the camera is configured to capture assessment movement video data of the one or more prescribed movements of the user while participating in the prescribed movement programs, wherein the interface is communicatively coupled to the network;
one or more physiological sensors communicatively coupled to the network configured to obtain physiological data from a user;
one or more processing means to execute instruction on a memory to perform the following operations:
initiating a prescribed movement program containing at least one or more movements to be performed by a user;
providing a user a unique code for accessing the apparatus;
providing information to a remote user about one or more exercise routine established by a third-party that can be accessed via a digital communication network by the pre-determined user to which the pre-determined exercise routine corresponds;
providing an interface including a display and a camera, wherein the display allows user to access the exercise routine and the camera is configured to capture video data of the one or more movements of the user while participating in the exercise routine;
receiving from the user a selected exercise routines for broadcasting on the display;
providing the selected exercise routine and communication through the interface;
receiving video data of the one or more movements of the pre-determined user;
analyzing the one or more movements with respect to a movement database and one or more pre-determined movement parameters assigned to the pre-determined user with a modeling artificial intelligence guided algorithm, wherein the modeling algorithm comprises a human pose estimation model for each movement using artificial intelligence-guided modelling algorithm;
monitoring and recording the movements of the user utilizing a modeling algorithm with a camera;
generating feedback based upon the movement of the user and analysis of the user movement;
communicating response feedback to the user wherein the feedback includes at least one of the following:
generated real-time feedback using artificial intelligence from the system comparing a measured movement parameter to a target movement parameter or
generated real-time feedback using visual, auditory, haptic modalities;
communicating the post-performance feedback to the third-party.
